(19) United States
(12) Patent Application Publication
(10) Pub. No.: US 2015/0322176 A1
(43) Pub. Date: Nov. 12, 2015

(54) NOVEL POLYSACCHARIDE IMMUNOGENS FROM CLOSTRIDIUM BOLTEAE ISOLATED FROM AUTISTIC SUBJECTS AND METHODS AND USES THEREOF

(71) Applicant: University of Guelph, Guelph (CA)

(72) Inventor: Mario Artur Monteiro, Guelph (CA)

(21) Appl. No.: 14/688,491

(22) Filed: Apr. 16, 2015

Related U.S. Application Data
(60) Provisional application No. 61/980,296, filed on Apr. 16, 2014.

(51) Int. Cl. C08B 37/00; A61K 47/48; C12N 7/00; A61K 39/08

(52) U.S. Cl. CPC C08B 37/0087; A61K 39/08; A61K 47/4833; C12N 7/00; C12N 2710/16131

(57) ABSTRACT

The disclosure relates to Clostridium bolteae cell surface polysaccharides, compositions comprising Clostridium bolteae cell surface polysaccharides, and includes kits, methods and uses thereof.

### RELATED APPLICATIONS

This application claims the benefit of U.S. Provisional Application No. 61/980,296 filed on Apr. 16, 2014, which is incorporated herein in its entirety.

### FIELD

The present disclosure relates to novel immunogenic cell surface polysaccharides and methods and uses thereof.

### BACKGROUND

Individuals with autistic spectrum disorders (ASDs) are likely to experience chronic gastrointestinal (GI) irritations, such as constipation and diarrhea [1]. GI disorders have a demonstrably high correlation with ASD and current literature suggests that it occurs with rates as high as 91.4% of patients [2]. Of the ASD patients affected, 75.6% suffer from diarrhea [2]. Diarrheal irritations can be extremely uncomfortable for a child and have been noted to have a loose association with regressive autism [1]. This correlation could represent specificity for the regressive autism subset disorder, and diarrheal irritations, as a whole, could be linked to the difference in GI flora of autistic children when compared to control samples [1]. Changes in commensal bacteria can have adverse effects on the affected individual and some species can produce neurotoxins which can induce clinical infections [2]. Select species produce potent exotoxins and Clostridium is a bacterial genus that contains several of the most toxigenic species known [2].

Species of the Clostridium genus produce short-chain fatty acids (SCFAs) as end products of metabolism, such as butyrate, propionate and acetate, that have the ability to alter the motility and contraction rate of the GI tract [3], for example through reduction of the amplitude of antral contractions as well as increase in contraction frequency [4]. Therefore, changes in the abundance of Clostridium spp. in the GI microbiota could potentially affect the GI function of an individual. It has previously been shown in independent studies that the GI microbiota of autistic children contained an over-representation of Clostridium spp., compared to healthy neurotypical controls [1,2,5] and in particular Finegold et al. found an overabundance of C. bolteae in fecal samples from their autistic subjects compared to controls [1]. C. bolteae is a Gram-positive bacterium whose growth requires an anaerobic environment with an optimal temperature of 37° C. such as that found in the human gut [6]. It is possible that chronic diarrheal episodes associated with some forms of ASD could be attributed to an overabundance of C. bolteae, and/or related to as-yet uncharacterized toxins produced by this and related bacterial species.

Vancomycin is a glycopeptide antimicrobial with specific activity against Gram-positive bacteria. When orally administered, this compound is poorly absorbed by host tissues and instead accumulates in the GI tract; in fact this property makes vancomycin the treatment of choice for management of diarrhea caused by pathogenic clostridia such as C. difficile [7]. In a small proof-of-principle intervention trial, vancomycin was orally administered to severely autistic children with chronic, persistent diarrhea, and short-term improvement of symptoms was seen in 8 out of 10 children [1,8,9]. However, these improvements were not persistent, and once vancomycin was withdrawn, any behavioral gains in these children were lost; in turn suggesting that microbial dysbiosis was suppressed but not eliminated. Prolonged use of vancomycin is not encouraged due to the threat of increased incidence and emergence of vancomycin-resistant pathogens [6]. Bacterial cell-wall capsular polysaccharides (CPSs) vaccines are being developed against Campylobacter jejuni [10] and C. difficile [11,12].

### SUMMARY

The present disclosure provides a novel Clostridium bolteae cell surface polysaccharide including its covalent chemical structures. This novel polysaccharide may be used in immunogenic compositions, in anti-C. bolteae vaccine preparations and/or as diagnostic markers.

Accordingly, the present disclosure includes an isolated immunogenic Clostridium bolteae cell surface polysaccharide.

In one embodiment, the cell surface polysaccharide comprises repeating disaccharide units of the formula I:

→3)-α-D-Manp-(1→4)-β-D-Rhap-(1→ (I)

wherein Man is mannose, Rha is rhamnose and p is pyranose.

In another embodiment, the cell surface polysaccharide is a compound of the formula II:

[→3)-α-D-Manp-(1→4)-β-D-Rhap-(1→]n (II)

wherein n is an integer from 1 to 1000, Man is mannose, Rha is rhamnose and p is pyranose.

In another embodiment, n is an integer from 1 to 100, 2 to 100 or 25 to 100.

In a further embodiment, the cell surface polysaccharide is conjugated to a carrier molecule. In one embodiment, the carrier molecule is BSA, CRM₁₉₇, MIEP, Diphtheria toxoid, Tetanus toxoid, KLH or proteins derived from Bordetella.

In yet another embodiment, the cell surface polysaccharide is obtained by growing Clostridium bolteae bacteria in suitable medium, separating bacterial cells from the medium, extracting cell surface polysaccharides by mild acid treatment under conditions to cleave the polysaccharides from the cell surface material, and purifying the extracted cell surface polysaccharides.

In another aspect, the disclosure provides an immunogenic composition comprising the cell surface polysaccharide disclosed herein and a pharmaceutically acceptable excipient, carrier, buffer, stabilizer, or mixtures thereof. In one embodiment, the immunogenic composition further comprises an immunostimulatory component, such as an adjuvant.

In yet another aspect, the disclosure provides a vaccine composition comprising the cell surface polysaccharide disclosed herein and a pharmaceutically acceptable excipient, carrier, buffer, stabilizer, or mixtures thereof. In one embodiment, the vaccine composition further comprises an immunostimulatory component, such as an adjuvant.

In yet a further aspect, the disclosure provides a kit comprising the cell surface polysaccharide disclosed herein, the immunogenic composition disclosed herein, or the vaccine composition disclosed herein, and instructions for the use thereof.

In another aspect, the disclosure provides a method of inducing an immune response against Clostridium bolteae in a subject, comprising administering to said subject an effective amount of a cell surface polysaccharide disclosed herein. Also provided is use of an effective amount of a cell surface polysaccharide disclosed herein for inducing an immune response against Clostridium bolteae in a subject in need thereof. Further provided is use of an effective amount of a cell surface polysaccharide disclosed herein in the preparation of a medicament for inducing an immune response against Clostridium bolteae in a subject in need thereof. Also provided is an effective amount of a cell surface polysaccharide disclosed herein for use in inducing an immune response against Clostridium bolteae in a subject in need thereof.

In another embodiment, there is provided a method of treating or preventing Clostridium bolteae infection in a subject, comprising administering to said subject an effective amount of a cell surface polysaccharide disclosed herein. Also provided is use of an effective amount of a cell surface polysaccharide disclosed herein for treating or preventing Clostridium bolteae infection in a subject in need thereof. Further provided is use of an effective amount of a cell surface polysaccharide disclosed herein in the preparation of a medicament for treating or preventing Clostridium bolteae infection in a subject in need thereof. Also provided is an effective amount of a cell surface polysaccharide disclosed herein for use in treating or preventing Clostridium bolteae infection in a subject in need thereof.

Even further provided is a method of treating or preventing Clostridium bolteae-associated gastrointestinal symptoms in a subject, comprising administering to said subject an effective amount of a cell surface polysaccharide disclosed herein. Also provided is use of an effective amount of a cell surface polysaccharide disclosed herein for treating or preventing Clostridium bolteae-associated gastrointestinal symptoms in a subject in need thereof. Further provided is use of an effective amount of a cell surface polysaccharide disclosed herein in the preparation of a medicament for treating or preventing Clostridium bolteae-associated gastrointestinal symptoms in a subject in need thereof. Also provided is an effective amount of a cell surface polysaccharide disclosed herein for use in treating or preventing Clostridium bolteae-associated gastrointestinal symptoms in a subject in need thereof.

In another embodiment, the present disclosure provides a method of inducing an immune response against Clostridium bolteae in a subject, comprising administering to said subject an effective amount of an immunogenic composition disclosed herein. Also provided is a use of an immunogenic composition disclosed herein for inducing an immune response against Clostridium bolteae in a subject in need thereof. Further provided is a use of an immunogenic composition disclosed herein in the preparation of a medicament for inducing an immune response against Clostridium bolteae in a subject in need thereof. Also provided is an immunogenic composition disclosed herein for use in inducing an immune response against Clostridium bolteae in a subject in need thereof.

In yet another embodiment, there is provided a method of treating or preventing Clostridium bolteae infection in a subject, comprising administering to said subject an effective amount of an immunogenic composition disclosed herein. Also provided is a use of an immunogenic composition disclosed herein for treating or preventing Clostridium bolteae infection in a subject in need thereof. Further provided is a use of an immunogenic composition disclosed herein in the preparation of a medicament for treating or preventing Clostridium bolteae infection in a subject in need thereof. Also provided is an immunogenic composition disclosed herein for use in treating or preventing Clostridium bolteae infection in a subject in need thereof.

In a further embodiment, there is provided a method of treating or preventing Clostridium bolteae-associated gastroinstestinal symptoms in a subject, comprising administering to said subject an effective amount of an immunogenic composition disclosed herein. Also provided is a use of an immunogenic composition disclosed herein for treating or preventing Clostridium bolteae-associated gastroinstestinal symptoms in a subject in need thereof. Further provided is a use of an immunogenic composition disclosed herein in the preparation of a medicament for treating or preventing Clostridium bolteae-associated gastroinstestinal symptoms in a subject in need thereof. Also provided is an immunogenic composition disclosed herein for use in treating or preventing Clostridium bolteae-associated gastroinstestinal symptoms in a subject in need thereof.

In yet another embodiment, the present disclosure provides a method of inducing an immune response against Clostridium bolteae in a subject, comprising administering to said subject an effective amount of a vaccine composition disclosed herein. Also provided is use of a vaccine composition disclosed herein for inducing an immune response against Clostridium bolteae in a subject in need thereof. Further provided is use of a vaccine composition disclosed herein in the manufacture of a medicament for inducing an immune response against Clostridium bolteae in a subject in need thereof. Also provided is a vaccine composition disclosed herein for use in inducing an immune response against Clostridium bolteae in a subject in need thereof.

In another embodiment, the disclosure provides a method of treating or preventing Clostridium bolteae infection in a subject, comprising administering to said subject an effective amount of a vaccine composition of disclosed herein. Also provided is use of a vaccine composition disclosed herein for treating or preventing Clostridium bolteae infection in a subject in need thereof. Further provided is use of a vaccine composition disclosed herein in the manufacture of a medicament for treating or preventing Clostridium bolteae infection in a subject in need thereof. Also provided is a vaccine composition disclosed herein for use in treating or preventing Clostridium bolteae infection in a subject in need thereof.

In a further embodiment, there is provided a method of treating or preventing Clostridium bolteae-associated gastrointestinal symptoms in a subject, comprising administering to said subject an effective amount of a vaccine composition disclosed herein. Also provided is use of a vaccine composition disclosed herein treating or preventing Clostridium bolteae-associated gastrointestinal symptoms in a subject in need thereof. Further provided is use of a vaccine composition disclosed herein in the manufacture of a medicament for treating or preventing Clostridium bolteae-associated gastrointestinal symptoms in a subject in need thereof. Also provided is a vaccine composition disclosed herein for use in treating or preventing Clostridium bolteae-associated gastrointestinal symptoms in a subject in need thereof.

In one embodiment, the subject is a human.

In another embodiment, the subject has autism spectrum disorder or is at risk for autism spectrum disorder. In an embodiment, the subject is an autistic child or a child at risk of autism.

In yet another embodiment, the gastrointestinal symptoms comprise diarrhea. In another embodiment, the gastrointestinal symptoms comprise constipation.

In a further aspect, the present disclosure includes a method of detecting Clostridium bolteae in a test sample comprising assaying the sample for the presence of an isolated cell surface polysaccharide disclosed herein.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows (A) ¹H NMR spectrum of C. bolteae CPS showing two anomeric resonances (denoted A for α-Man and B for β-Rha) and the remainder of the ring proton resonances; (B) 1D ¹H-¹H NOESY showing the strong inter-nOe connectivity between H-1 (A1) of Man and H-4 (B4) of Rha and a weaker inter-nOe connectivity between A1 and B6,6′.

FIG. 2 shows a contour plot of the most dominant dihedral angles of the glycosidic linkages observed during the molecular dynamic simulation of the modeled trisaccharide α-D-Man-(1→4)-β-D-Rha-(1→3)-α-D-Man: (A) α-D-Man-(1→4)-β-D-Rha and (B) β-D-Rha-(1→3)-α-D-Man.

FIG. 3 shows an immunodot blot of raw C. bolteae CPS with antisera from rabbits inoculated with native C. bolteae CPS.

FIG. 4 shows (A) and (B) the covalent chemical structures of the cell surface polysaccharide isolated from C. bolteae. Also shown are the through-space ¹H-¹H nOe connectivities observed in ¹H NMR nOe experiments, which revealed the conformations present in C. bolteae cell surface polysaccharide.

### DETAILED DESCRIPTION

### I. Definitions

The term “Clostridium bolteae” as used herein includes all strains of Clostridium bolteae, including for example, strains 16351 and 14578 that were isolated form autistic children.

The term “isolated” as used herein refers to Clostridium bolteae cell surface polysaccharides substantially free of bacterial cell material or extraction solvent when produced from growing bacterial strains of Clostridium bolteae.

As used herein the term “Clostridium bolteae cell surface polysaccharide” includes those isolated from bacterial strains of Clostridium bolteae, and also includes polysaccharides produced synthetically to have the same structure and/or composition of a Clostridium bolteae cell surface polysaccharides disclosed herein. “Produced synthetically” includes for example, cell surface polysaccharides produced from techniques such as recombinant DNA technology, genetic knockout mice and/or chemical synthesis.

The term “covalent chemical structure” as used herein means the chemical formula for a compound where all groups are linked via covalent bonds.

As used herein the term “immunogenic” means the ability to elicit an immune response.

As used herein the term “vaccine” refers to a composition that prevents Clostridium bolteae infection, treats Clostridium bolteae infection and/or reduces shedding of Clostridium bolteae.

The term “therapeutically effective amount”, “effective amount” or “sufficient amount” means a quantity sufficient to, when administered to the subject, including a mammal, for example a human, achieve a desired result, for example an amount effect to elicit an immune response in a subject. Effective amounts of therapeutic may vary according to factors such as the disease state, age, sex, weight of the animal. Dosage or treatment regime may be adjusted to provide the optimum therapeutic response.

Moreover, a “treatment” regime of a subject with a therapeutically effective amount may consist of a single administration, or alternatively comprise a series of applications. The length of the treatment period depends on a variety of factors, such as the severity of the disease and/or symptoms, the age of the patient, the concentration and the activity of the polysaccharides, or a combination thereof. It will also be appreciated that the effective dosage of the compound used for the treatment or prevention may increase or decrease over the course of a particular treatment or prevention regime. Changes in dosage may result and become apparent by standard diagnostic assays known in the art. The compounds of the present disclosure may be administered before, during or after exposure to the bacteria.

The expression “biologically compatible form in vivo” as used herein means a form of the substance to be administered in which any toxic effects are outweighed by the therapeutic effects.

The term “eliciting an immune response” or “inducing an immune response” as used herein means initiating, triggering, causing, enhancing, improving or augmenting any response of the immune system, for example, of either a humoral or cell-mediated nature. The initiation or enhancement of an immune response can be assessed using assays known to those skilled in the art including, but not limited to, antibody assays (for example ELISA assays), antigen specific cytotoxicity assays and the production of cytokines (for example ELISPOT assays).

The term “subject” as used herein refers to any member of the animal kingdom, optionally a mammal, including humans. In one embodiment, the subject is a human. In yet another embodiment, the subject has autism or is at risk for autism. In a further embodiment, the subject is a child with autism or a child at risk for autism.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

For example, the phrase “treating or preventing Clostridium bolteae infection” includes inhibiting Clostridium bolteae infection, preventing Clostridium bolteae infection, decreasing the severity of Clostridium bolteae infection, inhibiting Clostridium bolteae colonization, reducing shedding of Clostridium bolteae, preventing Clostridium bolteae colonization or improving signs and symptoms related to Clostridium bolteae infection and the phrase “treating or preventing Clostridium bolteae-associated gastrointestinal symptoms” includes inhibiting Clostridium bolteae-associated gastrointestinal symptoms, preventing Clostridium bolteae-associated gastrointestinal symptoms, decreasing the severity of Clostridium bolteae-associated gastrointestinal symptoms or improving signs and symptoms related to having Clostridium bolteae-associated gastrointestinal symptoms. The present disclosure also includes the treatment or prevention of any disease that is associated with a Clostridium bolteae infection.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### II. Compounds and Compositions

As mentioned above, the present disclosure describes the isolation and identification of the covalent chemical structure of a Clostridium bolteae cell surface polysaccharide. This novel polysaccharide is exposed on the cell surface of Clostridium bolteae and may be used in immunogenic compositions, carbohydrate-based vaccine preparations and/or as diagnostic markers.

Accordingly, the present disclosure includes an isolated immunogenic Clostridium bolteae cell surface polysaccharide.

The present disclosure also includes an immunogenic composition comprising an isolated immunogenic Clostridium bolteae cell surface polysaccharide.

The present disclosure further includes a vaccine composition comprising a Clostridium bolteae cell surface polysaccharide.

The present disclosure describes the isolation and characterization of a distinct cell surface Clostridium bolteae cell surface polysaccharide.

Using chemical analysis and analytical techniques, such as gas chromatography (GC), nuclear magnetic resonance (NMR) and mass spectrometry (MS), it has been determined that the cell surface polysaccharide of Clostridium bolteae is a polymer of repeating disaccharide units linked through phosphodiester bonds. Specifically, the cell surface polysaccharide was found to be a repeating unit composed of mannose and rhamnose.

In an embodiment, the cell surface polysaccharide is obtained by isolation from strains of Clostridium bolteae bacteria, for example by growing Clostridium bolteae bacteria in suitable medium, separating bacterial cells from the medium, extracting cell surface polysaccharides by mild acid treatment under conditions to cleave the polysaccharides from the cell surface material, and purifying the extracted cell surface polysaccharides. In a further embodiment the mild acid is 0.1% to 5%, suitably 2% acetic acid. In a further embodiment the cell surface polysaccharides are purified or separated by centrifugation, size exclusion chromatography and/or anion exchange chromatography.

In one embodiment, the cell surface polysaccharide comprises repeating disaccharide units of the formula I:

→3)-α-D-Manp-(1→4)-β-D-Rhap-(1→ (I)

wherein Man is mannose, Rha is rhamnose and p is pyranose.

In another embodiment, the cell surface polysaccharide is a compound of the formula II:

[→3)-α-D-Manp-(1→4)-β-D-Rhap-(1→]n (II)

wherein n is an integer from 1 to 1000, Man is mannose, Rha is rhamnose and p is pyranose.

In another embodiment, n in Formula II is an integer from 1 to 100, 2 to 100, 10 to 100, or 25 to 100.

In another embodiment, the present disclosure provides an immunogenic composition comprising a Clostridium bolteae cell surface polysaccharide and a pharmaceutically acceptable excipient, carrier, buffer, stabilizer, or mixtures thereof.

In yet another embodiment, the present disclosure provides an immunogenic composition comprising a Clostridium bolteae cell surface polysaccharide and an adjuvant.

In one embodiment, the immunogenic composition comprises a cell surface polysaccharide comprising repeating disaccharide units of the formula I:

→3)-α-D-Manp-(1→4)-β-D-Rhap-(1→ (I)

wherein Man is mannose, Rha is rhamnose and p is pyranose.

In another embodiment, the immunogenic composition comprises a cell surface polysaccharide of the formula II:

[→3)-α-D-Manp-(1→4)-β-D-Rhap-(1→]n (II)

wherein n is an integer from 1 to 1000, Man is mannose, Rha is rhamnose and p is pyranose.

In another embodiment, n is an integer from 1 to 100, 2 to 100, 10 to 100, or 25 to 100.

In another embodiment, the present disclosure provides a vaccine composition comprising a Clostridium bolteae cell surface polysaccharide and a pharmaceutically acceptable excipient, carrier, buffer, stabilizer, or mixtures thereof.

In yet another embodiment, the present disclosure provides a vaccine composition comprising a Clostridium bolteae cell surface polysaccharide and an adjuvant.

In one embodiment, the vaccine composition comprises a cell surface polysaccharide comprising repeating disaccharide units of the formula I:

→3)-α-D-Manp-(1→4)-β-D-Rhap-(1→ (I)

wherein Man is mannose, Rha is rhamnose and p is pyranose.

In another embodiment, the vaccine composition comprises a cell surface polysaccharide of the formula II:

[→3)-α-D-Manp-(1→4)-β-D-Rhap-(1→]n (II)

wherein n is an integer from 1 to 1000, Man is mannose, Rha is rhamnose and p is pyranose.

In another embodiment, n is an integer from 1 to 100, 2 to 100, 10 to 100, or 25 to 100.

Glycoconjugate vaccines are known to enhance the immunogenic properties of carbohydrates. Hence, by coupling Clostridium bolteae cell surface polysaccharides to a carrier molecule, it is possible to maximize the immunogenic response of the carbohydrate-based vaccine.

Accordingly, in another embodiment of the present disclosure there is included a Clostridium bolteae cell surface polysaccharide disclosed herein conjugated to a carrier molecule.

In an embodiment, the cell surface polysaccharide comprising repeating disaccharide units of the formula I are conjugated to a carrier molecule. In another embodiment, the cell surface polysaccharide of formula II is conjugated to a carrier molecule.

Another embodiment of the present disclosure is an immunogenic composition comprising a Clostridium bolteae cell surface polysaccharide disclosed herein conjugated to a carrier molecule.

A further embodiment of the present disclosure is a vaccine composition comprising a Clostridium bolteae cell surface polysaccharide disclosed herein conjugated to a carrier molecule.

In one embodiment, the carrier molecule is a protein. In another embodiment, the carrier molecule is bovine serum albumin (BSA). In another embodiment, the carrier molecule is cross reactive material, for example, CRM₁₉₇. CRM₁₉₇ is a nontoxic version of a Diphtheria toxin that has been successfully used in pneumococcal conjugate vaccines (Anderson, P. W., 1983, Infect. Immun. 39:233-238). In another embodiment, the carrier molecule is MIEP (major immunoenhancing protein). MIEP may be derived from the outer membrane complex of Neisseria meningitis type B and other meningococcal group B (Merck). In another embodiment, the carrier molecule is Diphtheria toxoid. In a further embodiment, the carrier molecule is Tetanus toxoid. In another embodiment, the carrier molecule is a protein derived from Bordetella. In yet a further embodiment, the carrier molecule is keyhole limpet hemocyanin (KLH).

The carrier molecule may be attached to the cell surface polysaccharide using known methods. For example, via an ester or amide linkage between available hydroxy or carboxy groups on the saccharides and carboxyl or amine groups on the protein.

Other carrier molecules and methods of their attachment have been previously reported (see for example U.S. Pat. No. 4,673,574, the contents of which are incorporated herein by reference).

Immunogenicity can be significantly improved if the immunizing agent (i.e. Clostridium bolteae cell surface polysaccharide or immunogenic compositions comprising the Clostridium bolteae cell surface polysaccharide or vaccine compositions comprising the Clostridium bolteae cell surface polysaccharides disclosed herein) is regardless of administration format, co-immunized with an immunostimulatory component, such as an adjuvant. Adjuvants enhance the immunogenicity of an immunogen but are not necessarily immunogenic in and of themselves. Adjuvants may act by retaining the immunogen locally near the site of administration to produce a depot effect facilitating a slow, sustained release of immunogen to cells of the immune system. Adjuvants can also attract cells of the immune system to an immunogen depot and stimulate such cells to elicit immune response. As such, embodiments of this present disclosure encompass compositions including for example immunogenic, vaccine or pharmaceutical compositions further comprising adjuvants.

Another aspect of the present disclosure is an immunogenic composition comprising a Clostridium bolteae cell surface polysaccharide disclosed herein and an immunostimulatory component, such as an adjuvant.

Another aspect of the present disclosure is a vaccine composition comprising a Clostridium bolteae cell surface polysaccharide disclosed herein and an immunostimulatory component, such as an adjuvant.

Adjuvants have been used for many years to improve the host immune responses to, for example, vaccines. Intrinsic adjuvants (such as lipopolysaccharides) normally are the components of killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Some of these adjuvants are toxic, however, and can cause undesirable side-effects making them unsuitable for use in humans and many animals. Indeed, only aluminum hydroxide and aluminum phosphate (collectively commonly referred to as alum) are routinely used as adjuvants in human and veterinary vaccines. The efficacy of alum in increasing antibody responses to Diphtheria and Tetanus toxoids is well established.

A wide range of extrinsic adjuvants can provoke potent immune responses to immunogens. These include saponins complexed to membrane protein antigens (immune stimulating complexes), pluronic polymers with mineral oil, killed mycobacteria and mineral oil, Freund's complete adjuvant, KLH, bacterial products such as muramyl dipeptide (MDP) and lipopolysaccharide (LPS), as well as lipid A, and liposomes.

In one aspect of the present disclosure, adjuvants useful in any of the embodiments described herein are as follows. Adjuvants for parenteral immunization include aluminum compounds (such as aluminum hydroxide, aluminum phosphate, and aluminum hydroxy phosphate). The antigen can be precipitated with, or adsorbed onto, the aluminum compound according to standard protocols. Other adjuvants such as RIBI (ImmunoChem, Hamilton, Mont.) can also be used in parenteral administration.

Adjuvants for mucosal immunization include bacterial toxins (e.g., the cholera toxin (CT), the E. coli heat-labile toxin (LT), the Clostridium difficile toxin A and the pertussis toxin (PT), or combinations, subunits, toxoids, or mutants thereof). For example, a purified preparation of native cholera toxin subunit B (CTB) can be of use. Fragments, homologs, derivatives, and fusion to any of these toxins are also suitable, provided that they retain adjuvant activity. Preferably, a mutant having reduced toxicity is used. Suitable mutants have been described (e.g., in WO 95/17211 (Arg-7-Lys CT mutant), WO 96/6627 (Arg-192-Gly LT mutant), and WO 95/34323 (Arg-9-Lys and Glu-129-Gly PT mutant)). Additional LT mutants that can be used in the methods and compositions disclosed herein include, for example Ser-63-Lys, Ala-69-Gly, Glu-110-Asp, and Glu-112-Asp mutants. Other adjuvants (such as a bacterial monophosphoryl lipid A (MPLA) of various sources (e.g., E. coli, Salmonella minnesota, Salmonella typhimurium, or Shigella flexneri, saponins, or polylactide glycolide (PLGA) microspheres) can also be used in mucosal administration.

Adjuvants useful for both mucosal and parenteral immunization include polyphosphazene (for example, WO 95/2415), DC-chol (3 b-(N-(N′,N′-dimethyl aminomethane)-carbamoyl) cholesterol (for example, U.S. Pat. No. 5,283,185 and WO 96/14831) and QS-21 (for example, WO 88/9336).

A subject may be immunized with a composition including for example an immunogenic, vaccine or pharmaceutical composition comprising a Clostridium bolteae cell surface polysaccharide disclosed herein by any conventional route as is known to one skilled in the art. This may include, for example, immunization via a mucosal (e.g., ocular, intranasal, oral, gastric, pulmonary, intestinal, rectal, vaginal, or urinary tract) surface, via the parenteral (e.g., subcutaneous, intradermal, intramuscular, intravenous, or intraperitoneal) route or intranodally. Preferred routes depend upon the choice of the immunogen as will be apparent to one skilled in the art. The administration can be achieved in a single dose or repeated at intervals. The appropriate dosage depends on various parameters understood by skilled artisans such as the immunogen itself (i.e. peptide vs. nucleic acid (and more specifically type thereof), the route of administration and the condition of the animal to be vaccinated (weight, age and the like).

The Clostridium bolteae cell surface polysaccharide or immunogenic compositions or vaccine compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The pharmaceutical composition may be supplied, for example but not by way of limitation, as a lyophilized powder which is reconstituted with sterile water or saline prior to administration to the patient.

Compositions including for example immunogenic, vaccine or pharmaceutical compositions of the present disclosure may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

The compositions disclosed herein can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

Accordingly, another embodiment of the present disclosure is a pharmaceutical composition comprising an effective amount of a Clostridium bolteae cell surface polysaccharide disclosed herein in admixture with a suitable excipient, diluent, carrier, buffer or stabilizer.

In a suitable embodiment, the pharmaceutical compositions are suitable for administration to subjects in a biologically compatible form in vivo.

Another aspect of the present disclosure is a kit comprising the cell surface polysaccharide disclosed herein, the immunogenic compositions disclosed herein, the vaccine compositions disclosed herein or the pharmaceutical compositions disclosed herein, and instructions for use thereof.

The kit can also include ancillary agents. For example, the kit can include an instrument for injecting the immunogenic composition of the present disclosure into a subject, such as a syringe; a vessel for storing or transporting the immunogenic composition; and/or pharmaceutically acceptable excipients, carriers, buffers or stabilizers.

### III. Methods and Uses

Another aspect of the present disclosure is a method of inducing an immune response against Clostridium bolteae in a subject by administering to the subject an effective amount of a Clostridium bolteae cell surface polysaccharide disclosed herein.

A further aspect of the present disclosure is a method of treating or preventing Clostridium bolteae infection in a subject by administering to the subject an effect amount of a Clostridium bolteae cell surface polysaccharide disclosed herein.

An additional aspect of the present disclosure is a method of treating or preventing Clostridium bolteae-associated gastrointestinal symptoms in a subject by administering to the subject an effective amount of a Clostridium bolteae cell surface polysaccharide disclosed herein.

The present disclosure also provides uses of a Clostridium bolteae cell surface polysaccharide disclosed herein to induce an immune response against Clostridium bolteae in a subject, to treat or prevent Clostridium bolteae infection in a subject, and to treat or prevent Clostridium bolteae-associated gastrointestinal symptoms in a subject.

Other aspects of the present disclosure include uses of a Clostridium bolteae cell surface polysaccharide disclosed herein for the manufacture of a medicament to induce an immune response against Clostridium bolteae in a subject, to treat or prevent Clostridium bolteae infection, and to treat or prevent Clostridium bolteae-associated gastrointestinal symptoms.

Further aspects of the present disclosure include a Clostridium bolteae cell surface polysaccharide disclosed herein for use in inducing an immune response against Clostridium bolteae in a subject, for use in treating or preventing Clostridium bolteae infection, and for use in treating or preventing Clostridium bolteae-associated gastrointestinal symptoms.

Another aspect of the present disclosure is a method of inducing an immune response against Clostridium bolteae in a subject by administering to the subject an effective amount of the immunogenic compositions disclosed herein where the immunogenic composition comprises a Clostridium bolteae cell surface polysaccharide disclosed herein and optionally an adjuvant.

A further aspect of the present disclosure is a method of treating or preventing Clostridium bolteae infection in a subject by administering to the subject an effective amount of the immunogenic compositions disclosed herein where the immunogenic composition comprises a Clostridium bolteae cell surface polysaccharide disclosed herein.

An additional aspect of the present disclosure is a method of treating or preventing Clostridium bolteae-associated gastrointestinal symptoms in a subject by administering to the subject an effective amount of the immunogenic compositions disclosed herein where the immunogenic composition comprises a Clostridium bolteae cell surface polysaccharide disclosed herein.

The present disclosure also provides uses of the immunogenic compositions disclosed herein where the immunogenic composition comprises a Clostridium bolteae cell surface polysaccharide disclosed herein to induce an immune response against Clostridium bolteae in a subject, to treat or prevent Clostridium bolteae infection in a subject, and to treat or prevent Clostridium bolteae-associated gastrointestinal symptoms in a subject.

Other aspects of the present disclosure include uses of the immunogenic compositions disclosed herein where the immunogenic composition comprises a Clostridium bolteae cell surface polysaccharide disclosed herein for the manufacture of a medicament to induce an immune response in a subject against Clostridium bolteae, to treat or prevent Clostridium bolteae infection, and to treat or prevent Clostridium bolteae-associated gastrointestinal symptoms.

Further aspects of the present disclosure include the immunogenic compositions disclosed herein where the immunogenic composition comprises a Clostridium bolteae cell surface polysaccharide disclosed herein for use in inducing an immune response in a subject against Clostridium bolteae, for use in treating or preventing Clostridium bolteae infection, and for use in treating or preventing Clostridium bolteae-associated gastrointestinal symptoms.

Another aspect of the present disclosure is a method of inducing an immune response against Clostridium bolteae in a subject by administering to the subject an effective amount of the vaccine compositions disclosed herein where the vaccine composition comprises a Clostridium bolteae cell surface polysaccharide disclosed herein.

A further aspect of the present disclosure is a method of treating or preventing Clostridium bolteae infection in a subject by administering to the subject an effective amount of the vaccine compositions disclosed herein where the vaccine composition a Clostridium bolteae cell surface polysaccharide disclosed herein.

An additional aspect of the present disclosure is a method of treating or preventing Clostridium bolteae-associated gastrointestinal symptoms in a subject by administering to the subject an effective amount of the vaccine compositions disclosed herein where the vaccine composition comprises one or more of the Clostridium bolteae cell surface polysaccharides disclosed herein.

The present disclosure also provides uses of the vaccine compositions disclosed herein where the vaccine composition comprises a Clostridium bolteae cell surface polysaccharide disclosed herein to induce an immune response against Clostridium bolteae in a subject, to treat or prevent Clostridium bolteae infection in a subject, and to treat or prevent Clostridium bolteae-associated gastrointestinal symptoms in a subject.

Other aspects of the present disclosure include uses of the vaccine compositions disclosed herein where the vaccine composition comprises a Clostridium bolteae cell surface polysaccharide disclosed herein for the manufacture of a medicament to induce an immune response against Clostridium bolteae in a subject, to treat or prevent Clostridium bolteae infection, and to treat or prevent Clostridium bolteae-associated gastrointestinal symptoms.

Further aspects of the present disclosure include the vaccine compositions disclosed herein where the vaccine composition comprises a Clostridium bolteae cell surface polysaccharide disclosed herein for use in inducing an immune response against Clostridium bolteae in a subject, for use in treating or preventing Clostridium bolteae infection, and for use in treating or preventing Clostridium bolteae-associated gastrointestinal symptoms.

The methods and uses of the present disclosure are applicable to subjects including human beings. In a particular embodiment, the subject is autistic or is at risk of autism. In another embodiment, the subject is an autistic child or is a child at risk of autism.

The present disclosure also includes methods and uses of the Clostridium bolteae cell surface polysaccharide as a diagnostic marker for a Clostridium bolteae infection. For example, since Clostridium bolteae uniquely possess the cell surface polysaccharide disclosed herein, the presence of this polysaccharide in its cellular material can indicate the presence of this bacteria in a sample. The sample may be from a human or animal subject or from food or water, or other substance, suspected of infection with Clostridium bolteae.

Accordingly the present disclosure includes a method of detecting Clostridium bolteae in a test sample comprising assaying the sample for the presence of an isolated cell surface polysaccharide disclosed herein. The disclosure also includes the use of an isolated cell surface polysaccharide disclosed herein to detect Clostridium bolteae in a test sample.

The presence of an isolated cell surface polysaccharide disclosed herein may be assayed, for example, by isolating the polysaccharide from the sample and performing chemical analyses to determine the identity of the saccharides that are present in the polysaccharide. Such chemical analyses can include one or more of (i) GLC-MS of the corresponding alditol acetates, MS and NMR spectroscopy.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Materials and Methods

### Bacterial Strains and Growth Conditions

C. bolteae strains 16351 and 14578 were originally isolated from feces from an autistic subject [5]. Strains were cultured on fastidious anaerobe agar (Lab90) supplemented with 5% defibrinated sheep's blood at 37° C. in a reduced atmosphere (N₂:CO₂:H₂ 80:10:10) for 36-48 hr before harvesting by scraping from plates using microbiological loops. The cells were washed in sterile phosphate buffered saline, pelleted by centrifugation and subsequently freeze-dried.

Extraction and Purification of C. bolteae Polysaccharide

Bacterial cell pellets were stirred for 3 hr at 70° C. in a hot water-phenol solution amenable to the method of Westphal and Jann [13] and similar to that used by us to extract C. difficile polysaccharides [14]. The aqueous layer was subjected to dialysis (3500 Da) against deionized water and size exclusion chromatography (Bio-Gel P2). No detectable amounts of RNA or protein were observed.

### Monosaccharide Analysis

Alditol acetate derivatives of the CPS for composition analysis were produced [15]. The material first was subjected to hydrolysis by 4 M trifluoroacetic acid at 105° C. for 4.5 hr, which was followed by overnight reduction using NaBD₄ in H₂O at room temperature. The resulting product was peracetylated using acetic anhydride at 105° C. for 1.5 hr. The alditol acetates derivatives were analyzed by gas chromatography-mass spectrometry (GC-MS). Linkage analysis was performed by analysis of the permethylated alditol acetates by GC-MS [16]. GC-MS analyses were performed on a PolarisQ MS with Trace Ultra GC system (Thermo Electron Corporation) in the electron impact mode with a DB-17 MD capillary column.

### Nuclear Magnetic Resonance Spectroscopy

1D and 2D NMR, ¹H-¹H TOCSY (Total Correlation Spectroscopy), ¹H-¹H NOESY (Nuclear Overhauser-Effect Spectroscopy), ¹H-¹³C HSQC (Heteronuclear Single-Quantum Correlation Spectroscopy) and ¹H-¹³C HMBC (Heteronuclear Multiple Bond Correlation Spectroscopy) spectra were recorded using a Bruker NSC 600 MHz spectrometer. The 1D ³¹P NMR spectra was recorded using a Bruker Mac 400 MHz spectrometer. For the 1D TOCSY experiment the mixing time was set to 160 ms and for the 2D NOESY experiment the mixing time was set to 300 ms. Samples were lyophilized three times with D₂O (99%) prior to NMR experiments. External references were used in all experiments; TSP (δ_{H} 0.00, δ_{C} 0.0) in D₂O for both ¹H and ¹³C, and orthophosphoric acid (δ_{O} 0.0) in D₂O for ³¹P.

### Molecular Dynamics

The molecular dynamic simulation was performed using the AMBER force field with GLYCAM_{—}06h parameters which were designed for carbohydrates [17,18]. The software package AMBER 10 was used to create the trisaccharide, [α-D-Man-(1→4)-β-D-Rha-(1→3)-α-D-Man, followed by energy minimizing calculations using an implicit solvation with the first 1000 cycles being the steepest decent followed by conjugate gradient energy minimization until the convergence criteria of dᵣₘₛ=0.0001 kcal/mol·Å was reached. The molecular dynamic simulation was done using an implicit solvent at 37° C. with a dielectric multiplicative constant set to 78.5. Non-bonded and electrostatic interactions scaling factors were set to 1.0. The system was heated from 5 to 310 K in 25 ps and maintained at 310 K for 50 ns using a weak-coupling algorithm. A cutoff of 100 Å was used for non-bonded interactions. Bonds containing hydrogen were constrained to their equilibrium lengths using the SHAKE algorithm. The dihedral angles, phi (O₅—C₁—O′ₓ—C′ₓ) and psi (C₁—O′ₓ—C′ₓ—C′ₓ₋₁), were obtained using the program PTRAJ, part of the AMBER 10 software package, and the data was processed and displayed in GNUplot.

### Immunological Studies

New Zealand rabbits were immunized 4 times (every 10 days) subcutaneously with 200 μg of the purified C. bolteae polysaccharide and Complete Freund's Adjuvant. After 35 days, serum was extracted and used for immunotesting. For immunodot blots, 2 μl of 1 mg/ml C. bolteae purified polysaccharide solution (or its corresponding systematic dilutions) were applied onto nitrocellulose membrane. Within the limits of detection, no detectable protein or RNA/DNA was observed. The membrane was blocked with 5% milk powder (w/v) in Tris-Buffered Saline solution with 0.05% Tween20 (v/v) (TBS-T) buffer. The blocked membrane was then incubated for 2 h with the rabbit serum at 1:2000 fold dilution. After washes in TBS-T buffer, the membrane was incubated with a horseradish peroxidase-coupled goat-anti-IgG rabbit antibody at 1:2000 dilution for one hour. After washing, the membrane was incubated with TMB precipitating stain solution (ScyTek). Imaging was performed with the BioRad XR+ imager.

Results and Discussion

Elucidation of C. bolteae Cell-Wall Capsular Polysaccharide

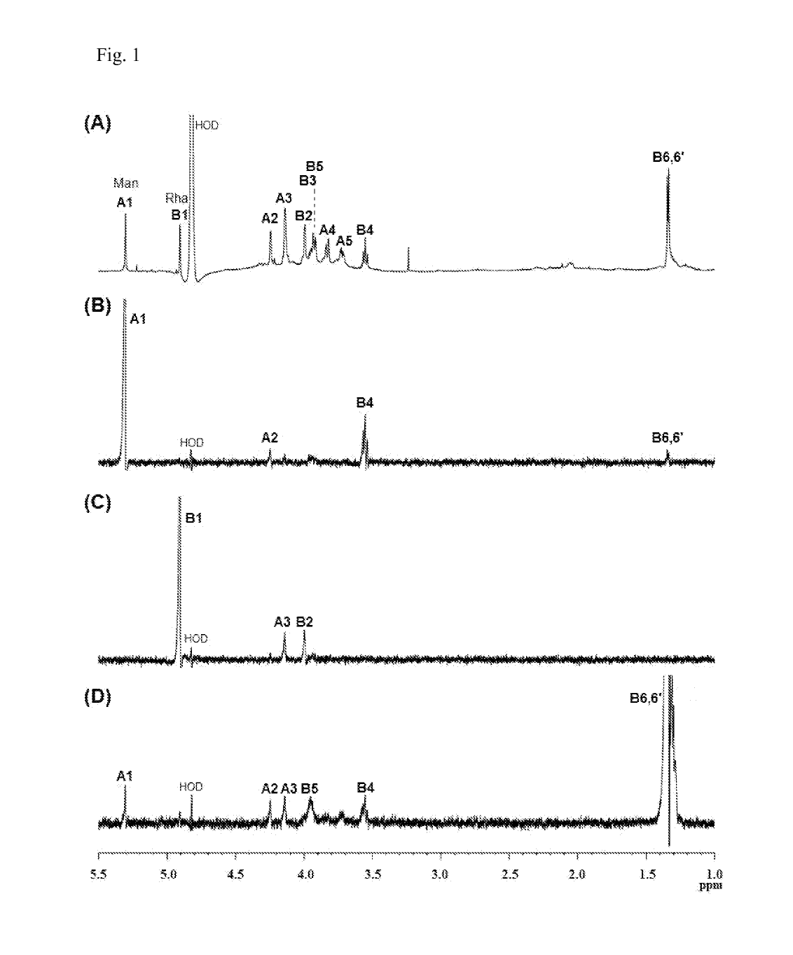

The monosaccharide composition analysis revealed that rhamnose (Rha) and mannose (Man) comprised the C. bolteae CPS, and with the Man present as a 3-monosubstituted pyranose unit and the Rha as a 4-monosubstituted pyranose unit. The ¹H NMR spectra of the CPS (FIG. 1A) displayed two anomeric resonances (A and B), consistent with the number of linkages observed in the GC-MS analysis. 2D ¹H-¹H COSY, TOCSY, and ¹H-¹³C HSQC experiments assisted in the assignment of the ring proton and carbon resonances (Table 1). The observed vicinal coupling allowed for the assignment of the Manp and Rhap ring-proton systems, in which the selective magnetization of the 6-deoxy resonances (1.34 ppm) were key to unlock the proton resonances of the Rha unit. Anomeric resonance A (α) was assigned to the Man residue and B (β) to the Rha unit.

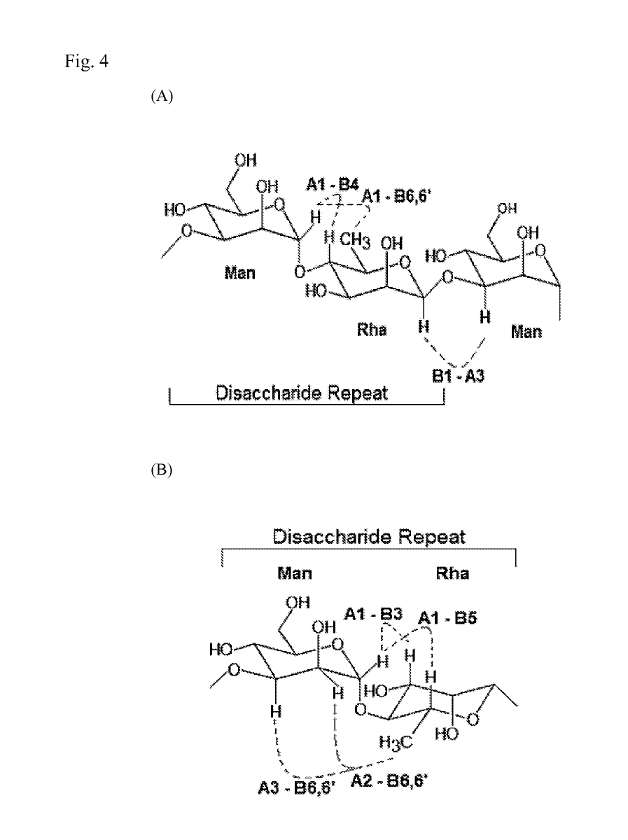

The alternating sequence of the monosaccharide residues with a disaccharide repeating block was confirmed through a ¹H-¹³C HMBC experiment with noted correlations between H-1 of A and C-4 of B for a [α-Man-(1→4)-β-Rha] sequence, and H-1 of B and C-3 of A for a [β-Rha-(1→3)-α-Man] sequence. In addition, ¹H-¹H NOESY corroborated the HMBC findings with inter-NOE connectivities being observed between H-1 (δ6.30) of the 3-substituted α-Man (A1) and H-4 (δ3.55) of the 4-substituted β-Rha (B) [α-Manp-(1→4)-β-Rhap] (FIG. 1B), and between H-1 (δ4.90) of the 4-substituted β-Rhap (B) and H-3 (δ4.13) of the 3-substituted α-Man (A) [β-Rhap(1→3)-α-Manp] (FIG. 1C) (See FIG. 4(A)).

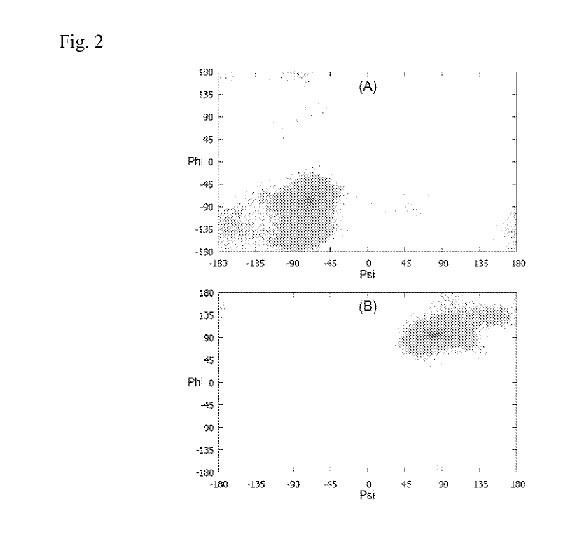

A molecular dynamics study revealed that the glycosidic bond orientations shown above were indeed the dominant conformations present in the CPS based on the occurrences of the position of the dihedral angles observed in the modeled trisaccharide Man(1→4)Rha(1→3)Man (FIG. 2). However, the NOESY experiment also showed one strong inter-NOE connectivity between H-2 of Man and H-6,6′ of Rha, and two others of less intensity between H-1 of Man and H-3/H5 of Rha, which revealed an additional conformational arrangement about the α-Man-(1→4)-β-Rha glycosidic linkage within the CPS conformation (FIG. 1D) (see FIG. 4(B)).

Immunogenicity of C. bolteae Cell-Wall Capsular Polysaccharide

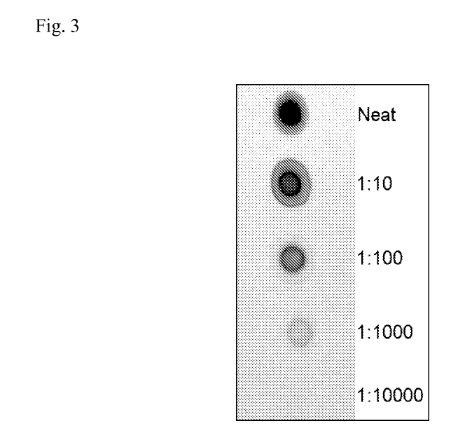

Immunodot blots with the rabbit serum indicated strong interaction of the antibodies with the CPS (FIG. 3). Even at a dilution of 1:1000 (i.e. 2 ng) of the CPS, strong interaction was observed. In contrast, no staining was observed with a similarly purified cell-wall polysaccharide (PSII) from C. difficile demonstrating specificity of the immune response. The results suggest that the isolated CPS from C. bolteae presents a sufficiently immunogenic target for the development of an efficient C. bolteae vaccine and diagnostic target.

### Discussion

C. bolteae was found to produce a CPS comprised of disaccharide repeating blocks composed α-D-Manp and β-D-Rhap units: [→3)-α-D-Manp-(1→4)-β-D-Rhap-(1→]n. This C. bolteae CPS is markedly different from those of C. difficile [19] and C. perfringens [20,21], which manufacture CPSs that contain higher order oligosaccharide repeating blocks. The expression of D-Rha is of particular interest in that in nature the dominant form is L-Rha generated from UDP-D-Glc [22]. Previously, D-Rha has been found in the lipopolysaccharide (A-band) of Pseudomonas aeruginosa, in which D-Rha is biosynthesized from GDP-D-Man [23,24]. Since Man is a CPS component of C. bolteae, similar biosynthetic mechanisms may also be available to C. bolteae to furnish the D-Rha residue from GDP-Man.

The recent discovery of the conserved C. difficile cell-wall immunogenic polysaccharide [19] has attracted much attention and is now being evaluated as an anti-C. difficile vaccine by several groups [11,14, 25]. It follows that the immunogenic CPS described in this study can be evaluated as a vaccine immunogen to manage the population of C. bolteae in the intestines of autistic children with chronic gastrointestinal ailments, and may prevent the augmentation of regressive-autism related symptoms. The C. bolteae CPS may also be used as a diagnostic marker to establish the degree of colonization in patients and thus help medical doctors in choosing the correct mode of treatment.

### Prophetic Example(s) of Preparing Conjugates

C. bolteae cell surface polysaccharides disclosed herein may be coupled to a stable carrier protein, such as, for example, CRM₁₉₇, KLH and/or Tetanus Toxoid, using coupling techniques known in the art to yield a glycoconjugate vaccine to treat or prevent C. bolteae infections, or to induce an immune response against C. bolteae.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
¹H and ¹³C NMR assignments of C. bolteae
16351. (δ, ppm; 25° C.)

| | H₁ | H₂ | H₃ | H₄ | H₅ | H_{6a/6b} |
|---|---|---|---|---|---|---|
| Residue | C₁ | C₂ | C₃ | C₄ | C₅ | C_{6a/6b} |
| α-Man (A) | 5.30 | 4.24 | 4.13 | 3.72 | 3.83 | 3.82/3.71 |
| | 111.1 | 76.8 | 83.4 | 72.4 | 65.3 | 65.3/65.2 |
| β-Rha (B) | 4.90 | 3.99 | 3.92 | 3.55 | 3.93 | 1.34 |
| | 103.6 | 73.2 | 72.8 | 82.1 | 70.6 | 19.8 |

### REFERENCES

[1] Finegold S M, Dowd S E, Gontcharova V, Liu C, Henley K E, et al. Pyrosequencing study of fecal microflora of autistic and control children. Anaerobe 2010; 16:444-453.
[2] Parracho H M, Bingman M O, Gibson G R, McCartney A L. Differences between the gut microflora of children with autistic spectrum disorders and that of healthy children. J Med Microbiol 2005; 54:987-991.
[3] MacFabe D F, Cain D P, Rodriguez-Capote K, Franklin A E, Hoffman J E, et al. Neurobiological effects of intraventricular propionic acid in rats: Possible role of short chain fatty acids on the pathogenesis and characteristics of autism spectrum disorders. Behav Brain Res 2007; 176:149-169.
[4] Cuche G, Malbert C H. Short-chain fatty acids present in the ileum inhibit fasting gastrointestinal motility in conscious pigs. Neurogastroent Motil 1999; 11:219-225.
[5] Finegold S M, Molitoris D, Song Y, Liu C, Vaisanen M-L, et al. Gastrointestinal Microflora Studies in Late-Onset Autism. Clin Infect Dis 2002; 35:S6-S16.
[6] Song Y, Liu C, Molitoris D R, Tomzynski T J, Lawson P A, et al. Clostridium bolteae sp. nov., Isolated from Human Sources. Syst Appl Microbiol 2003; 26:84-89.
[7] Venugopal A A, Johnson S. Current State of Clostridium difficile Treatment Options. Clin Infect Dis 2012; 55:S71-S76.
[8] Finegold S M, Downes J, Summanen P H. Microbiology of regressive autism. Anaerobe 2012; 18:260-262.
[9] Sandler R H, Finegold S M, Bolte E R, Buchanan C P, Maxwell A P, et al. Short-Term Benefit From Oral Vancomycin Treatment of Regressive-Onset Autism. J Child Neurol 2000; 15:429-435.
[10] Guerry P, Poly F, Riddle M, Maue A C, Chen Y-H, et al. Campylobacter polysaccharide capsules: virulence and vaccines. Frontiers Cell Infect Microbiol 2012; 2: doi: 10.3389/fcimb.2012.00007.
[11] Adamo R, Romano M R, Berti F, Leuzzi R, Tontini M, et al. Phosphorylation of the Synthetic Hexasaccharide Repeating Unit Is Essential for the Induction of Antibodies to Clostridium difficile PSII Cell Wall Polysaccharide. ACS Chem Biol 2012; 7:1420-1428.
[12] Monteiro M A, Ma Z, Bertolo L, Jiao Y, Arroyo L, et al. Carbohydrate-based Clostridium difficile vaccines. Exp Rev Vaccines. 2013; in press.
[13] Westphal O K, Jann K. Bacterial lipooligosaccharide. Extraction with phenol-water and further applications of the procedure. Meth Carbohydr Chem 1965; 5:83-91.
[14] Bertolo L, Boncheff A G, Ma Z, Chen Y-H, Wakeford T, Friendship R M, et al. Clostridium difficile carbohydrates: glucan in spores, PSII common antigen in cells, immunogenicity of PSII in swine and synthesis of a dual C. difficile—ETEC conjugate vaccine. Carbohydr Res 2012; 354:79-86.
[15] Sawardeker J S, Sloneker J H, Jeanes A. Quantitative Determination of Monosaccharides as Their Alditol Acetates by Gas Liquid Chromatography. Anal Chem 1965; 37:1602-1604.
[16] Ciucanu I, Kerek F. A simple and rapid method for the permethylation of carbohydrates. Carbohydr Res 1984; 131:209-217.
[17] Case D A, Darden T A, Cheatham T E, Simmerling C L, Wang J, Duke R E, et al. Amber 10. San Francisco: University of California 2008.
[18] Kirschner K N, Yongye A B, Tschampel S M, Daniels C R, Foley B L, Woods R J. GLYCAM06: A generalizable biomolecular force field. Carbohydrates. J Comp Chem 2008; 29:622-655.
[19] Ganeshapillai J, Vinogradov E, Rousseau J, Weese J S, Monteiro M A. Clostridium difficile cell-surface polysaccharides composed of pentaglycosyl and hexaglycosyl phosphate repeating units. Carbohydr Res 2008; 343:703-710.
[20] Kalelkar S, Glushka J, van Halbeek H, Morris L C, Cherniak R. Structure of the capsular polysaccharide of Clostridium perfringens Hobbs 5 as determined by NMR spectroscopy. Carbohydr Res 1997; 299:119-128.
[21] Sheng S, Cherniak R. Structure of the capsular polysaccharide of Clostridium perfringens Hobbs 10 determined by NMR spectroscopy. Carbohydr Res 1997; 305:65-72.
[22] Watt G, Leoff C, Harper A D, Bar-Peled M. A Bifunctional 3,5-Epimerase/4-Keto Reductase for Nucleotide-Rhamnose Synthesis in Arabidopsis. Plant Physiol 2004; 134:1337-1346.
[23] Rocchetta H L, Burrows L L, Lam J S. Genetics of O-Antigen Biosynthesis in Pseudomonas aeruginosa. Microbiol Mol Biol Rev 1999; 63:523-553.
[24] Ramm M, Wolfender J-L, Queiroz E F, Hostettmann K, Hamburger M. Rapid analysis of nucleotide-activated sugars by high-performance liquid chromatography coupled with diode-array detection, electrospray ionization mass spectrometry and nuclear magnetic resonance. J Chromatogr A 2004; 1034:139-148.
[25] Cappelletti A, Romano M R, Cakici O S, Adamo R, Berti F, Proietti D, et al. Conjugate vaccine against Clostridium difficile. Glycoconj J 2011; 28:265.
[26] Danieli E, Lay L, Proietti D, Berti F, Costantino P, Adamo R. First synthesis of C. difficile PS-II cell wall polysaccharide repeating unit. Org Lett 2011; 13:378-381.
[27] Oberli M A, Hecht M L, Bindschädler P, Adibekian A, Adam T, Seeberger P H. A Possible Oligosaccharide-Conjugate Vaccine Candidate for Clostridium difficile Is Antigenic and Immunogenic. Chem Biol 2011; 18:580-588.

1. An isolated immunogenic Clostridium bolteae cell surface polysaccharide comprising repeating disaccharide units of the formula (I):
→3)-α-D-Manp-(1→4)-β-D-Rhap-(1→ (I)
wherein Man is mannose, Rha is rhamnose and p is pyranose.

2. The cell surface polysaccharide of claim 1, wherein the cell surface polysaccharide is a compound of the formula II:
[→3)-α-D-Manp-(1→4)-β-D-Rhap-(1→]n (II)
wherein n is an integer from 1 to 1000, Man is mannose, Rha is rhamnose and p is pyranose.

3. The cell surface polysaccharide of claim 1, wherein n is an integer from 1 to 100.

4. The cell surface polysaccharide of claim 1, wherein the polysaccharide is conjugated to a carrier molecule.

5. The cell surface polysaccharide of claim 4, wherein the carrier molecule is BSA, CRM₁₉₇, MIEP, Diphtheria toxoid, KLH, Tetanus toxoid or proteins derived from Bordetella.

6. The cell surface polysaccharide of claim 1, obtained by growing Clostridium bolteae bacteria in suitable medium, separating bacterial cells from the medium, extracting cell surface polysaccharides by mild acid treatment under conditions to cleave the polysaccharides from the cell surface material, and purifying the extracted cell surface polysaccharides.

7. An immunogenic composition comprising the cell surface polysaccharide of claim 1 and a pharmaceutically acceptable excipient, carrier, buffer, stabilizer, or mixtures thereof.

8. The immunogenic composition of claim 7, further comprising an immunostimulatory component, such as an adjuvant.

9. A vaccine composition comprising the cell surface polysaccharide of claim 1 and a pharmaceutically acceptable excipient, carrier, buffer, stabilizer, or mixtures thereof.

10. The vaccine composition of claim 9, further comprising an immunostimulatory component, such as an adjuvant.

11. A method of inducing an immune response against Clostridium bolteae in a subject, comprising administering to said subject an effective amount of the cell surface polysaccharide of claim 1.

12. A method of treating or preventing Clostridium bolteae infection in a subject, comprising administering to said subject an effective amount of the cell surface polysaccharide of claim 1.

13. A method of treating or preventing Clostridium bolteae-associated gastrointestinal symptoms in a subject, comprising administering to said subject an effective amount of the cell surface polysaccharide of claim 1.

14. A method of inducing an immune response against Clostridium bolteae in a subject, comprising administering to said subject an effective amount of the immunogenic composition of claim 7.

15. A method of treating or preventing Clostridium bolteae infection in a subject, comprising administering to said subject an effective amount of the immunogenic composition of claim 7.

16. A method of treating or preventing Clostridium bolteae-associated gastroinstestinal symptoms in a subject, comprising administering to said subject an effective amount of the immunogenic composition of claim 7.

17. The method of claim 11, wherein the subject is a human being.

18. The method of claim 17, wherein the subject has autism or is at risk of autism.
